(19) United States
(12) Patent Application Publication
(10) Pub. No.: US 2011/0060250 A1
(43) Pub. Date: Mar. 10, 2011

(54) DEVICE FOR DETECTING VIBRATION IN LIVING BODY AND DEVICE FOR EXAMINING PHYSIOLOGICAL STATE OF THE LIVING BODY USING THE SAME

(76) Inventor: Akihiko Yanaga, Kanagawa (JP)

(21) Appl. No.: 12/922,886

(22) PCT Filed: May 10, 2007
(86) PCT No.: PCT/JP2007/059631

§ 371 Date: Sep. 16, 2010

(51) Int. Cl. A61B 5/103

(52) U.S. Cl. 600/587

(57) ABSTRACT

It is intended to provide a device for detecting a vibration in a living body by which a vibration caused by, for example, a vibration caused by a change in a part (in particular, the head) of a subject, respiration, pulsation, cough, snore, an eyeball movement, a body movement, a jaw movement or turn over can be detected; and a device for examining the physiological state of the living body using the same, and the device for detecting a vibration in the living body at least comprises: a housing on which a definite part (in particular, the head) of a subject is placed; a distortion plate provided on the inner upper face of the housing in contact with the upper face of the housing; a distortion-detection means bonded to the distortion plate; and a holding plate composed of a contacting part being in contact with the inner lower face of the housing and a holding part extending from the contacting part and the both edge of the distortion plate.

### BACKGROUND OF THE INVENTION

The present invention relates to a device for detecting a vibration in a living body using for detecting mainly physiological signals, for instance, respiration, pulsation, cough, snore, an eyeball movement, a body movement, a jaw movement, and turn over from a vibration caused by a change in a part, especially a head of a subject, and a device for examining physiological state of the living body using the same.

Patent document No. 1 discloses a device for respiration state which comprises an air mat arranged under a body of a subject lay down and pressure sensors for detecting pressure fluctuation inside this air mat and extracts and records physiological signals such as respiration frequency, heart rate and snoring signals and the like due to respiration movement in breathing muscle from output signals from the pressure sensors.

Patent document No. 2 discloses a panel for measuring physiological information. This panel has a floor plate part flexible elastically arranged under a subject and sensors for detecting distortion are installed on the floor plate part. In the state that the floor plate part is arranged under the subject, distortion of the floor plate part generated with physiological activity of the subject is detected by the sensors for detecting distortion. Besides, the floor plate part is put between elastic layers.

Patent document No. 1: JP 2000-271103 A

Patent document No. 2: JP 2006-43445 A

### DISCLOSURE OF THE INVENTION

### Problem to be Solved by the Invention

The device for detecting respiration state disclosed in the patent document No. 1 has a problem such that minute pressure variation cannot be detected because of detecting pressure variation of air inside the air mat arranged under the body of the subject. Besides, there is a problem such that it is difficult to maintain air pressure constantly inside the air mat.

Furthermore, in the device disclosed in the patent document No. 2, as the sensors for detecting distortion are arranged at both sides of the floor plate part put between the elastic layers with specific elastic force, minute variation is absorbed into the elastic layers, so that there is a problem such that only large vibration or variation can be detected.

Thus, the present invention is to provide a device for detecting a vibration in a living body using for detecting mainly physiological signals, for instance, respiration, pulsation, cough, snore, an eyeball movement, a body movement, a jaw movement, and turn over from a vibration caused by a change in a part, especially a head of a subject, and a device for examining physiological state of the living body using the same.

### Means for Solving Problem

Accordingly, the device for detecting a vibration in a living body is to be constituted of a housing on which a specific part, especially a head part of a subject is placed, a distortion plate provided on an inner upper surface of the housing so as to be in contact with the inner upper surface of the housing, a means for detecting distortion being in contact with the distortion plate, and a supporting plate at least constituted of a contact part being in contact with an inner lower surface of the housing and a supporting plate extending form the contact part in order to support both side ends of the distortion plate.

Due to this constitution, it is gone away to depend on a state of an installation surface where the means for detecting distortion is placed, and constant detection sensitivity can be obtained in long time usage of year unit. Besides, it is preferred that the means for detecting distortion is arranged on a middle part of the distortion plate, but preferably a plurality of means for detecting distortion are arranged along a longitudinal direction of the distortion plate. Furthermore, it is desirable that the means for detecting distortion is constituted of an electronic circuit board provided in the housing.

Besides, the distortion plate may have slit parts extending from an edge of the distortion plate to proximity of the means for detecting distortion.

Furthermore, the means for detecting distortion may be a piezo element (PZT, polyvinylidene fluoride) or a strain gauge, it is desirable that a material for collision prevention comprising an elastic material is arranged on the contact part of the supporting plate where the piezo element or the strain gauge faces.

Moreover, a device for detecting a vibration in a living body is constituted of at least a housing on which a head part of a subject is placed, a distortion plate is provided on an inner upper surface of the housing so as to be in contact with the inner upper surface of the housing, a first electrode fixed on the distortion plate, a supporting plate comprising a contact part being in contact with an inner lower surface of the housing and supporting parts extending from the contact part and supporting both edge parts of the distortion plate, and a second electrode provided on the contact part of the supporting plate on which the first electrode faces. Besides, it is desirable that capacitance is constituted of the first and the second electrodes.

Furthermore, a device for examining physiological state according to the present invention is to comprise a device for detecting a vibration in a living body according to the present invention, a means for detecting physiological signals sampling at least respiration waves, pulsation waves, snoring waves, body movement waves, eye movement waves, chewing movement waves or the like from vibration waves detected by the device for detecting a vibration in a living body, a means for judging apnea from the respiration waves and/or the snoring waves and further judging REM sleep from the chewing movement s waves and the eye movement waves.

Moreover, it is desirable that the distortion plate is made of resin or metal, and that the distortion plate has width within a range from 5 to 600 mm. length within a range from 10 to 1000 mm and thickness within a range from 0.1 to 30 mm. Besides, it is desirable that the supporting plate is provided with material and size corresponding to the distortion plate.

### EFFECT OF THE INVENTION

According to the device for detecting a vibration in a living body of the present invention, since the distortion plate being in contact with the upper surface of the housing on which the head of the subject is put is supported by the supporting plate being in contact with the lower surface of the housing, an effect such that minute vibration of the head of the subject can be transmitted to the means for detecting a vibration can be achieved.

Besides, because it becomes possible that the means for detecting a vibration can detect a minute vibration, it becomes possible to sample the waves showing respiration, pulsation, cough, snoring, body movement, eye movement, chewing movement or the like from the detected waves, and thus, it becomes possible to detect fine physiological state, especially apnea or REM sleep.

### BRIEF EXPLANATION OF DRAWINGS

FIG. 1 This is a cross-sectional view of a device for detecting a vibration in a living body according to a first embodiment of the present invention;

FIG. 2 This is a plan view showing one example of constitution of a supporting plate in the device for detecting a vibration in a living body according to the first embodiment of the present invention;

FIG. 3 This is a plan view showing constitution of a distortion plate in the device for detecting a vibration in a living body according to a first example of the present invention;

FIG. 4 This is a plan view showing constitution of a distortion plate in the device for detecting a vibration in a living body according to a second example of the present invention;

FIG. 5 This is a plan view showing constitution of a distortion plate in the device for detecting a vibration in a living body according to a third example of the present invention;

FIG. 6 This is a schematic diagram of an amplifier circuit,

FIG. 7 This is a schematic diagram of a device for examining physiological state according to the present invention,

FIG. 8 This is an illustration showing one example of signal treatment in MPU,

FIG. 9 This is a flow chart diagram showing an operation performed in MPU, and

FIG. 10 This is a cross-sectional view of a device for detecting a vibration in a living body according to a second embodiment of the present invention.

### EXPLANATIONS OF LETTERS OR NUMERALS

1 DEVICE FOR DETECTING A VIBRATION IN A LIVING BODY
2 HOUSING
3 DISTORTION PLATE
4 SENSOR FOR DETECTING DISTORTION
5 SUPPORTING PLATE
6 ELECTRONIC CIRCUIT SUBSTRATE
10 SLIT PART
20 AMPLIFIER CIRCUIT
30 DEVICE FOR EXAMINING PHYSIOLOGICAL STATE

### DESCRIPTION OF THE PREFERRED EMBODIMENTS

Hereinafter, the preferred embodiments are explained along the drawings.

### First Embodiment

A device for detecting a vibration in a living body 1 according to a first embodiment of the present invention is, for instance as shown in FIG. 1, constituted of at least a housing 2 on which a specific part, especially a head part of a subject is put, a distortion plate 3 provided on an inner upper surface 2A so as to be in contact with the inner upper surface 2A, a sensor for detecting distortion (concretely, a piezo element or distortion gauge) 4 fixed in an approximately middle part in the distortion plate 3, a supporting plate 5 supporting both end edge parts of the distortion plate 3, and an electronic circuit substrate 6 provided on the distortion plate 3 adjacent to the sensor for detecting distortion 4.

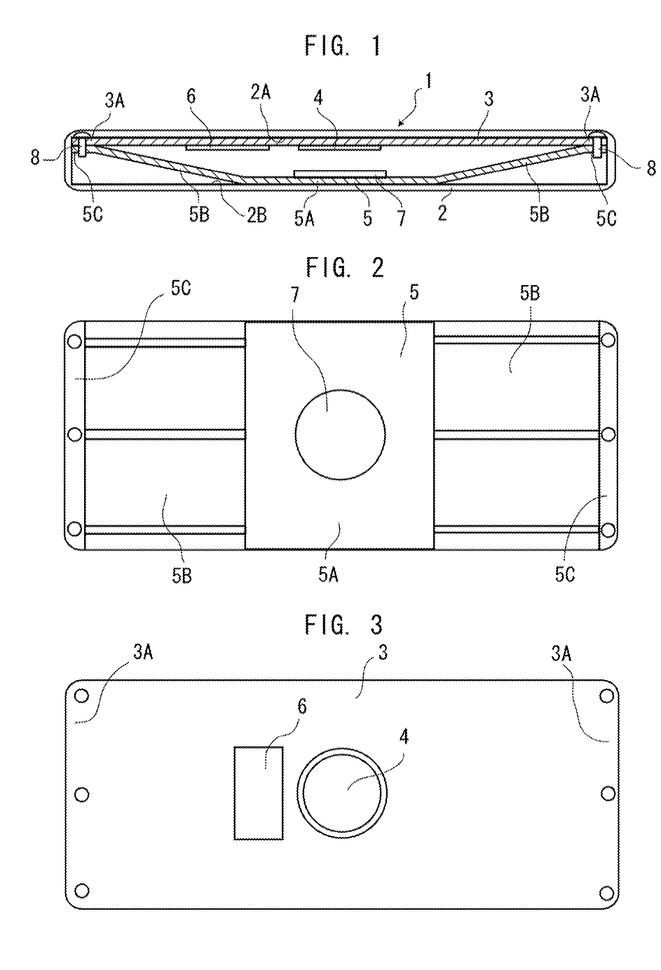

The supporting plate 5 is, for instance as shown in FIG. 2, a contact part 5A fixed or put in a middle of an inner lower surface 2B of the housing and supporting parts 5B extending from both sides of the contact part 5A to both end edge parts 3A of the distortion plate 3, and both edge parts 3A of the distortion plate 3 and fixed parts fixed by screws 8 or the like are formed on both end parts of the supporting parts 5B. Besides, a collision prevention material 7 made of an elastic material is arranged opposite to a sensor for detecting distortion 4. Furthermore, this supporting plate 5 is formed availably and selectively corresponding to a material and a shape of the following distortion plate 3.

In a first example as shown in FIG. 3, the distortion plate 3 is formed in an approximately square shape similar to a shape of the inner upper surface 2A of the housing 2 and formed of a material availably selected from groups consisting of metals such as aluminum, steel, stainless, brass, copper alloy, iron alloy, or the like, resins such as substrate for print wiring, plastics or the like, glass, and cloths, or a compound consisting of those materials, and it has a size that a head or a pillow can be put on, for instance, having width within a range from 5 mm to 600 mm, length within a range from 10 to 1000 mm of length and thickness within a range from 1 mm to 30 mm. Furthermore, in a first embodiment shown in FIG. 3, the sensor for detecting distortion 4 is fixed on an approximately center in a lower side surface of the distortion plate 3 and an electronic circuit substrate 6 corresponding to the sensor for detecting distortion 4 is arranged near the sensor for detecting distortion.

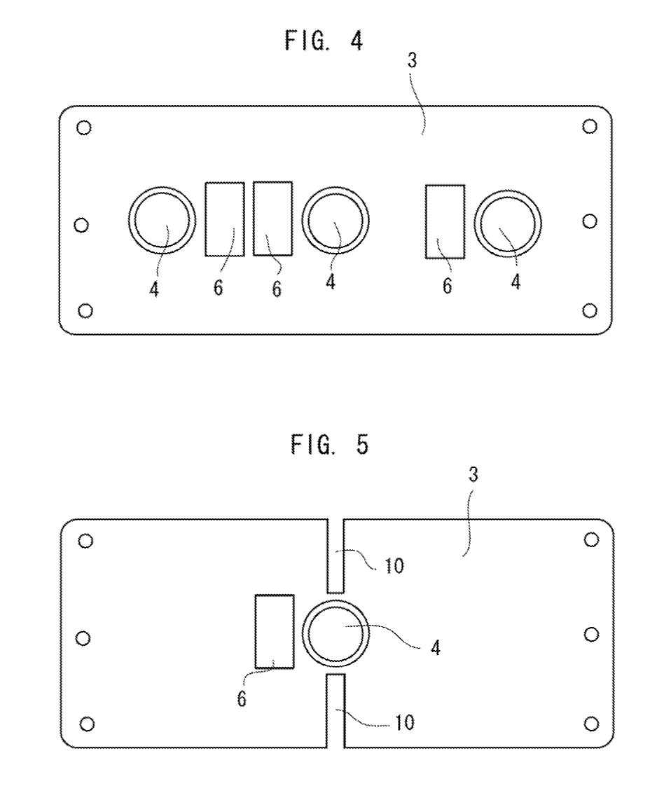

In a second example shown in FIG. 4, a plurality of sensors for detecting distortion 4 (three sensors in this embodiment) are arranged along a longitudinal direction of the distortion plate 3, more minute detection against a position of the head of the subject (not shown in figures) becomes possible.

In a third example shown in FIG. 5, slit parts 10 extending from side edges of longitudinal direction of the distortion plate 3 to proximities of the sensor for detecting distortion 4 respectively are provided. Thus, because strength of proximity of the sensor of detecting distortion 4 is decreased, it becomes easy to detect more minute vibration.

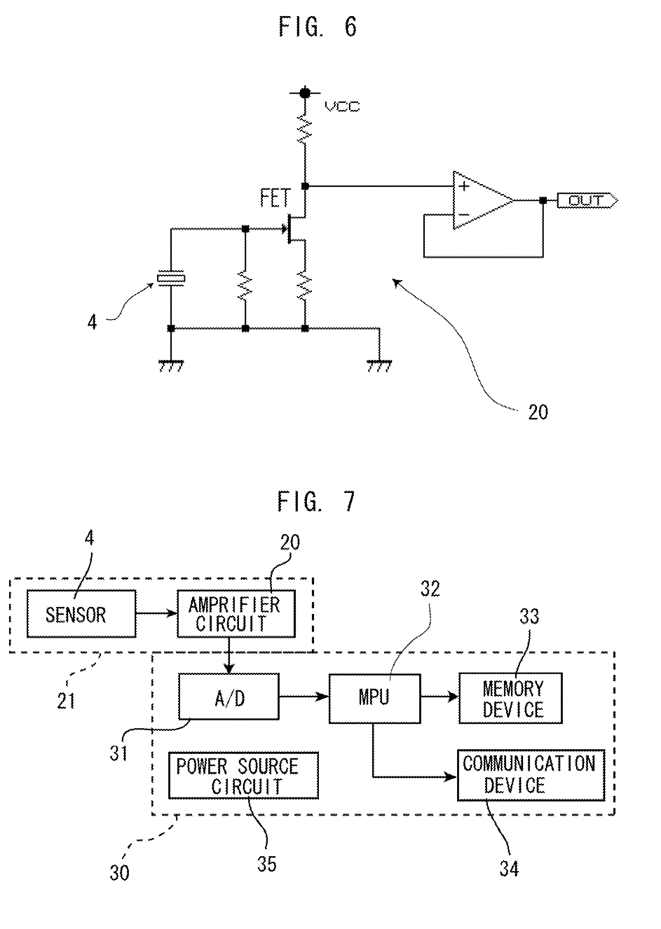

An amplifier circuit 20 as shown in FIG. 6 is provided in the electronic circuit substrate 6. This means that physical vibration on the distortion plate 3 is changed into electrical signals by the sensor for detecting distortion, amplified in the amplifier circuit 20 and output. This amplifier circuit 20 has well-known constitution.

In the device for detecting a vibration in a living body having the above constitution, because the hard distortion plate 3 is supported by the supporting plate 5 and thus specific tension applies to the distortion plate 3, a vibration generated from the head of the subject put on the housing 2 can be transmitted to the distortion plate 3 efficiently.

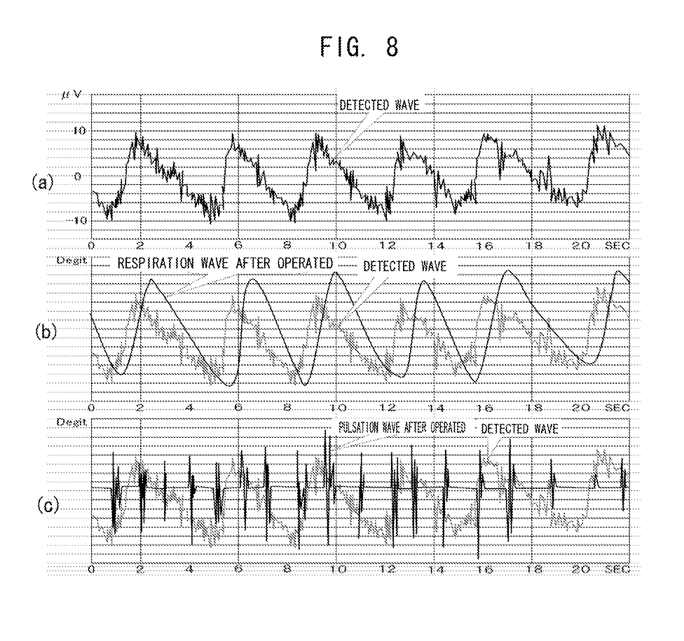

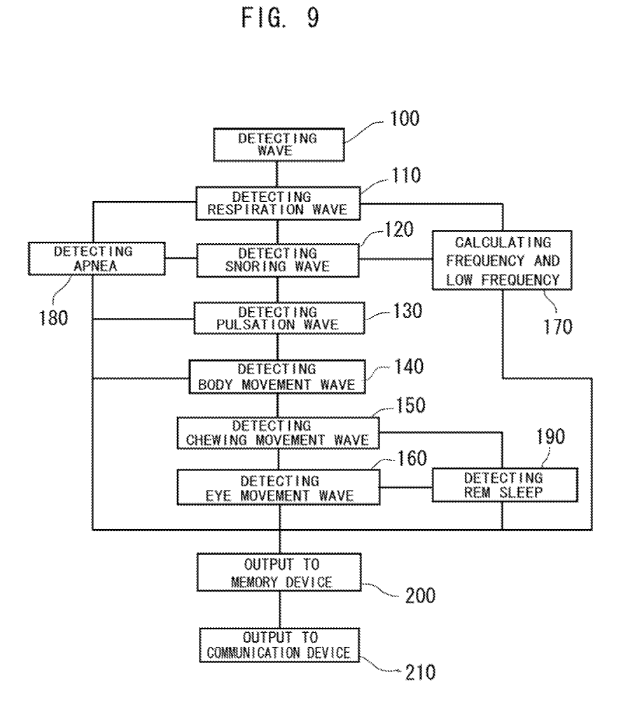

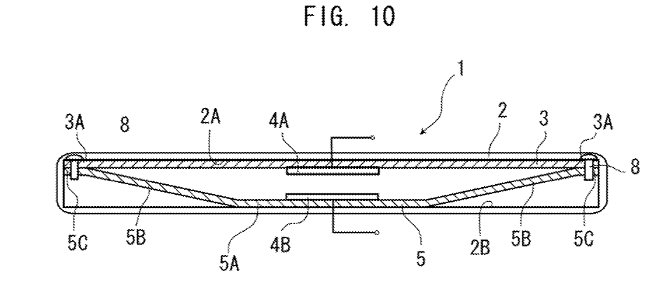

In a device for examining physiological state using the above mentioned device for detecting a vibration in a living body, for instance as shown in FIG. 7, a detecting wave (FIG. 8A) output form an electrical circuit 21 of the device for detecting a vibration in a living body constituted of the sensor 4 and the amplifier circuit 20 is transmitted to a micro processor unit (MPU) 32 via an A/D converter 31. In MPU 32, an operation, for instance, shown in a flowchart diagram in FIG. 9 is performed. Besides, the device for examining physiological state comprises at least a power source circuit 35, a memory device 33 and a communication device 34.

At step 100 in FIG. 9, the detected wave shown in FIG. 8A is changed to a digital signal via the A/D converter 31 in order to input into the MPU 32, and then signal treatment is performed as shown in FIG. 8B to pick up a respiration wave firstly as physiological signal wave. Similarly, at step 120, a snoring wave is picked up due to amplitude of the respiration wave or the like, and then at step 130, a pulsation wave is picked up as shown in FIG. 8C. Similarly, a body movement wave is picked up from amplitude of the detected wave, a variation period thereof and the like (step 140), a chewing movement wave is picked up from amplitude of the detected wave corresponding to jaw movement of the subject, a variation period thereof and the like (step 150), and further, an eye movement wave is picked up from more minute variation thereof (step 160).

Moreover, in the picked-up physiological signal waves, frequency of respiration and low frequency of respiration are detected from the respiration wave and the snoring wave at step 170, and it is detected at step 180 whether the subject is in apnea state or not. Besides, in this embodiment, it is detected whether the subject is in REM sleep due to the chewing movement wave and the eye movement wave or not.

The physiological data such as picked-up signal waves (the respiration wave, the snoring wave, the pulsation wave, the body movement wave, the chewing movement wave and the eye movement wave), calculated data (frequency of respiration, low frequency of respiration or the like) and the detected subject state (apnea, REM sleep) are output into the memory device 33 (step 200) and stored in it. Furthermore, the physiological data are output to the communication device 34 periodically (step 210) and then they are transmitted to necessary places such as an attending doctor's computer, a hospital, a caring center and the like.

### Second Embodiment

A device for detecting a vibration in a living body according to a second embodiment of the present invention shown in FIG. 10 comprises at least a housing 2 on which a head of a subject is put, a distortion plate 3 provided on an inner upper surface 2A of the housing 2 so as to be in contact with the inner upper surface 2A, a first electrode 4A fixed on the distortion plate 3, a supporting plate 5 comprising a contact part 5A being in contact with an inner lower surface 2B of the housing 2 and supporting parts 5B extending from the contact part 5A and supporting both end edge parts 3A of the distortion plate 3, and a second electrode 4B provided on the contact part 5A of the supporting plate 3 opposite to the first electrode 4A.

In this embodiment, by detecting variation of capacitance between the first electrode 4A and the second electrode 4B by the vibration of the distortion plate 3, the vibration can be detected as variation of an electrical signal. Also in this embodiment, the same operation as the first embodiment in the device for examining physiological state 30 is possible.

1-9. (canceled)

10. A device for detecting a vibration in a living body of a subject comprising at least:
a housing on which a head of the subject is put;
a distortion plate provided on an inner upper surface so as to be in contact with the inner upper surface;
a means for detecting distortion fixed on said distortion plate;
a supporting plate comprising a contact part fixed on an inner lower surface of the housing and supporting parts extending from the contact part and supporting both end edge parts of said distortion plate.

11. A device for detecting a vibration in a living body of a subject according to claim 10, wherein said means for detecting distortion comprises an electronic circuit substrate provided in said housing.

12. A device for detecting a vibration in a living body of a subject according to claim 10, wherein a plurality of the means for detecting distortion are arranged along a longitudinal direction of said distortion plate.

13. A device for detecting a vibration in a living body of a subject according to claim 11, wherein a plurality of the means for detecting distortion are arranged along a longitudinal direction of said distortion plate.

14. A device for detecting a vibration in a living body of a subject according to claim 10, wherein said distortion plate has slit parts extending from edges thereof to proximity of said means for detecting distortion.

15. A device for detecting a vibration in a living body of a subject according to claim 11, wherein said distortion plate has slit parts extending from edges thereof to proximity of said means for detecting distortion.

16. A device for detecting a vibration in a living body of a subject according to claim 12, wherein said distortion plate has slit parts extending from edges thereof to proximity of said means for detecting distortion.

17. A device for detecting a vibration in a living body of a subject according to claim 13, wherein said distortion plate has slit parts extending from edges thereof to proximity of said means for detecting distortion.

18. A device for detecting a vibration in a living body of a subject according to claim 10, wherein:
the means for detecting distortion is a piezo element or a distortion gauge, and
a collision preventing material is arranged on the contact part of said supporting plate which the piezo element or the distortion gauge faces.

19. A device for detecting a vibration in a living body of a subject according to claim 11, wherein:
the means for detecting distortion is a piezo element or a distortion gauge, and
a collision preventing material is arranged on the contact part of said supporting plate which the piezo element or the distortion gauge faces.

20. A device for detecting a vibration in a living body of a subject according to claim 12, wherein:
the means for detecting distortion is a piezo element or a distortion gauge, and
a collision preventing material is arranged on the contact part of said supporting plate which the piezo element or the distortion gauge faces.

21. A device for detecting a vibration in a living body of a subject according to claim 13, wherein:
the means for detecting distortion is a piezo element or a distortion gauge, and
a collision preventing material is arranged on the contact part of said supporting plate which the piezo element or the distortion gauge faces.

22. A device for detecting a vibration in a living body of a subject according to claim 14, wherein:
the means for detecting distortion is a piezo element or a distortion gauge, and
a collision preventing material is arranged on the contact part of said supporting plate which the piezo element or the distortion gauge faces.

23. A device for detecting a vibration in a living body of a subject according to claim 15, wherein:
the means for detecting distortion is a piezo element or a distortion gauge, and
a collision preventing material is arranged on the contact part of said supporting plate which the piezo element or the distortion gauge faces.

24. A device for detecting a vibration in a living body of a subject according to claim 16, wherein:
the means for detecting distortion is a piezo element or a distortion gauge, and
a collision preventing material is arranged on the contact part of said supporting plate which the piezo element or the distortion gauge faces.

25. A device for detecting a vibration in a living body of a subject according to claim 17, wherein:
the means for detecting distortion is a piezo element or a distortion gauge, and
a collision preventing material is arranged on the contact part of said supporting plate which the piezo element or the distortion gauge faces.

26. A device for examining physiological state comprising a device for detecting a vibration in a living body of a subject according to claim 10, further comprising at least:
a means for detecting physiological signals for picking up at least a respiration wave, a pulsation wave, a snoring wave, a body movement wave, an eye movement wave and a chewing movement wave from a vibration wave detected by said device for detecting a vibration in a living body of a subject, and
a means for judging apnea from the respiration wave and the snoring wave detected by said means for detecting physiological signals and for judging a REM sleep from the chewing movement wave and the eye movement wave.

27. A device for examining physiological state comprising a device for detecting a vibration in a living body of a subject according to claim 13, further comprising at least:
a means for detecting physiological signals for picking up at least a respiration wave, a pulsation wave, a snoring wave, a body movement wave, an eye movement wave and a chewing movement wave from a vibration wave detected by said device for detecting a vibration in a living body of a subject, and
a means for judging apnea from the respiration wave and the snoring wave detected by said means for detecting physiological signals and for judging a REM sleep from the chewing movement wave and the eye movement wave.

28. A device for examining physiological state comprising a device for detecting a vibration in a living body of a subject according to claim 17, further comprising at least:
a means for detecting physiological signals for picking up at least a respiration wave, a pulsation wave, a snoring wave, a body movement wave, an eye movement wave and a chewing movement wave from a vibration wave detected by said device for detecting a vibration in a living body of a subject, and
a means for judging apnea from the respiration wave and the snoring wave detected by said means for detecting physiological signals and for judging a REM sleep from the chewing movement wave and the eye movement wave.

29. A device for examining physiological state comprising a device for detecting a vibration in a living body of a subject according to claim 25, further comprising at least:
a means for detecting physiological signals for picking up at least a respiration wave, a pulsation wave, a snoring wave, a body movement wave, an eye movement wave and a chewing movement wave from a vibration wave detected by said device for detecting a vibration in a living body of a subject, and
a means for judging apnea from the respiration wave and the snoring wave detected by said means for detecting physiological signals and for judging a REM sleep from the chewing movement wave and the eye movement wave.
